(19) United States
(12) Patent Application Publication
(10) Pub. No.: US 2016/0016012 A1
(43) Pub. Date: Jan. 21, 2016

(54) STIMULATION APPARATUS USING LOW INTENSITY FOCUSED ULTRASOUND FOR PAIN MANAGEMENT AND MUSCLE STRENGTHENING

(71) Applicant: KOREA INSTITUTE OF SCIENCE AND TECHNOLOGY, Seoul (KR)

(72) Inventors: Inchan YOUN, Seoul (KR); Hyung Min KIM, Seoul (KR); Jinseok KIM, Seoul (KR); Kuiwon CHOI, Seoul (KR); Jong Woong PARK, Seoul (KR); Tae Young YUNE, Seoul (KR); Sungmin HAN, Seoul (KR); Yu Seon CHAE, Seoul (KR); Hoyoung JUNG, Seoul (KR)

(73) Assignee: KOREA INSTITUTE OF SCIENCE AND TECHNOLOGY, Seoul (KR)

(21) Appl. No.: 14/748,993

(22) Filed: Jun. 24, 2015

(30) Foreign Application Priority Data
Jul. 18, 2014 (KR) 10-2014-0091296

(51) Int. Cl. A61N 7/00; A61B 5/0488

(52) U.S. Cl. CPC A61N 7/00; A61B 5/0488; A61N 2007/0078; A61N 2007/0004

(57) ABSTRACT

A stimulation apparatus using low intensity focused ultrasound, which has a low intensity ultrasound focusing array having a plurality of transducers for outputting low intensity ultrasound beams, and a fixing device to which the low intensity ultrasound focusing array is attached, the fixing device being configured to fix the low intensity ultrasound focusing array to an upper body of a user. The low intensity ultrasound beams outputted from the transducers are focused to at least one focus. The focus is positioned to a spinal cord of the user or nerves around the spinal cord so that low intensity ultrasound stimulation is applied to the spinal cord or nerve cells of the nerves around the spinal cord.

### CROSS-REFERENCE TO RELATED APPLICATION

This application claims priority to Korean Patent Application No. 10-2014-0091296, filed on Jul. 18, 2014, and all the benefits accruing therefrom under 35 U.S.C. §119, the contents of which in its entirety are herein incorporated by reference.

### BACKGROUND

1. Field

The present disclosure relates to a stimulation apparatus using low intensity focused ultrasound, and more particularly, to a stimulation apparatus using low intensity focused ultrasound, which may apply low intensity focused ultrasound to a spinal cord or nerves around the spinal cord to manage a pain and strengthen muscles using the low intensity ultrasound stimulation.

Description about National Research and Development Support

This study was supported by the High-Tech Convergence Technology Development program of Ministry of Science, ICT and Future Planning, Republic of Korea (Project No. 1711005365) under the superintendence of National Research Foundation of Korea. This work was also partially supported by the Korea Health Technology R&D Project through the Korea Health Industry Development Institute (KHIDI), funded by the Ministry of Health & Welfare of the Republic of Korea (grant HI14C3477).

2. Description of the Related Art

In order to relieve a pain of a patient or strengthen muscles of the mobility impaired, it has been attempted to give electric stimulations to nerves or the like.

However, such an electric stimulation method requires inserting an electrode into a body of a patient, and secondary damages may be caused to the nerves or the like due to the electric stimulation.

For this reason, there is proposed a method using ultrasound which may pass through a human body.

Different from high intensity ultrasound which may cause necrosis due to high intensity, low intensity ultrasound is known to give medical effects without heating the human body or causing necrosis.

The unit of ultrasound intensity is classified into spatial-peak temporal-average intensity (Ispta) and spatial-peak pulse-average intensity (Isppa), based on the Acoustic Output Measurement Standard for Diagnostic Ultrasound Equipment of the NEMA (American Institute for Ultrasound in Medicine and National Electronics Manufacturers Administration).

Even though a standard for low intensity ultrasound is not yet clearly defined, in this disclosure, the “low intensity ultrasound” means ultrasound having a sound intensity lower than the spatial-peak temporal-average intensity (Ispta) of 3 W/cm² according to US FDA standards and European Safety standards, which does not damage a human body.

### SUMMARY

The present disclosure is directed to providing a stimulation apparatus using low intensity focused ultrasound, which may relieve a pain of a patient or strengthen muscles of the mobility impaired by non-invasively stimulating nerve cells using focused low intensity ultrasound without secondary damages.

In one aspect, there is provided a stimulation apparatus using low intensity focused ultrasound, which includes: a low intensity ultrasound focusing array having a plurality of transducers for outputting low intensity ultrasound beams; and a fixing device to which the low intensity ultrasound focusing array is attached, the fixing device being configured to fix the low intensity ultrasound focusing array to an upper body of a user, wherein the low intensity ultrasound beams outputted from the transducers are focused to at least one focus, and wherein the focus is positioned to a spinal cord of the user or nerves around the spinal cord so that low intensity ultrasound stimulation is applied to the spinal cord or nerve cells of the nerves around the spinal cord.

In an embodiment, the low intensity ultrasound beams may be focused to a single focus, and the position of the focus may be three-dimensionally moved by adjusting a phase difference of low intensity ultrasound generated by the transducers.

In an embodiment, low intensity ultrasound stimulation may be applied to a preset region of the spinal cord by moving the location of the focus continuously or intermittently, so as to find a point where a pain of the user decreases.

In an embodiment, the stimulation apparatus using low intensity focused ultrasound may store the point where the pain decreases, and if the low intensity ultrasound stimulation is completely applied to the preset region, the location of the focus may be moved to the point where the pain decreases, and then the pain of the user may be intensively treated or relieved.

In an embodiment, the stimulation apparatus using low intensity focused ultrasound may further include a controller which is operated by the user or another assistant to move the location of the focus.

In an embodiment, low intensity ultrasound stimulation may be applied to a spinal cord near a damaged spine or nerves around the spinal cord by moving the location of the focus continuously or intermittently, so as to assist muscle strengthening near the damaged spine.

In an embodiment, the fixing device may include a surface electromyogram detecting device for measuring electric activity of a muscle by means of surface electromyogram, and a muscle strengthening stimulation point where muscle strengthening is performed by the low intensity ultrasound may be found by using the surface electromyogram measured by the electromyogram detecting device.

In an embodiment, the found muscle strengthening stimulation point may be stored, and, if the muscle strengthening stimulation point is found, the location of the focus may be moved to the muscle strengthening stimulation point to intensively reinforce the muscle.

In an embodiment, the fixing device may be an abdominal binder surrounding an upper body of the user, and the abdominal binder may include a fixing portion to which the low intensity ultrasound focusing array is attached, and adhering portions extending from both ends of the fixing portion and capable of being coupled to each other.

In an embodiment, the fixing portion may have a bent shape, and at the fixing portion, the transducers of the low intensity ultrasound focusing array may be arranged to emit low intensity ultrasound beams toward one point.

### BRIEF DESCRIPTION OF THE DRAWINGS

FIGS. 1 to 3 are diagrams for illustrating a stimulation apparatus using low intensity focused ultrasound according to an embodiment of the present disclosure.

FIG. 4 is a diagram showing an ultrasound stimulation apparatus according to another embodiment of the present disclosure.

FIG. 5 is a diagram for illustrating a transducer according to an embodiment of the present disclosure.

FIG. 6 is a diagram for illustrating a piezoelectric effect of a piezoelectric element.

FIGS. 7 and 8 are diagrams showing an ultrasound beam focused by the stimulation apparatus using low intensity focused ultrasound according to an embodiment of the present disclosure.

FIGS. 9 and 10 are diagrams showing that a focus of the low intensity ultrasound beam is adjusted at the stimulation apparatus using low intensity focused ultrasound according to an embodiment of the present disclosure.

FIG. 11 is a diagram showing that a pain is controlled by stimulating a spinal cord using the stimulation apparatus using low intensity focused ultrasound according to an embodiment of the present disclosure.

FIG. 12 is a diagram showing that low intensity ultrasound stimulation is applied to nerve cells around a damaged spine using the stimulation apparatus using low intensity focused ultrasound according to an embodiment of the present disclosure.

FIG. 13 is a diagram showing an ultrasound stimulation apparatus according to another embodiment of the present disclosure.

### DETAILED DESCRIPTION

Hereinafter, preferred embodiments of the present disclosure will be described with reference to the accompanying drawings. Even though the present disclosure is described based on the embodiments depicted in the drawings, this is just an example, and the technical features, essential configuration and operations of the present disclosure are not limited thereto.

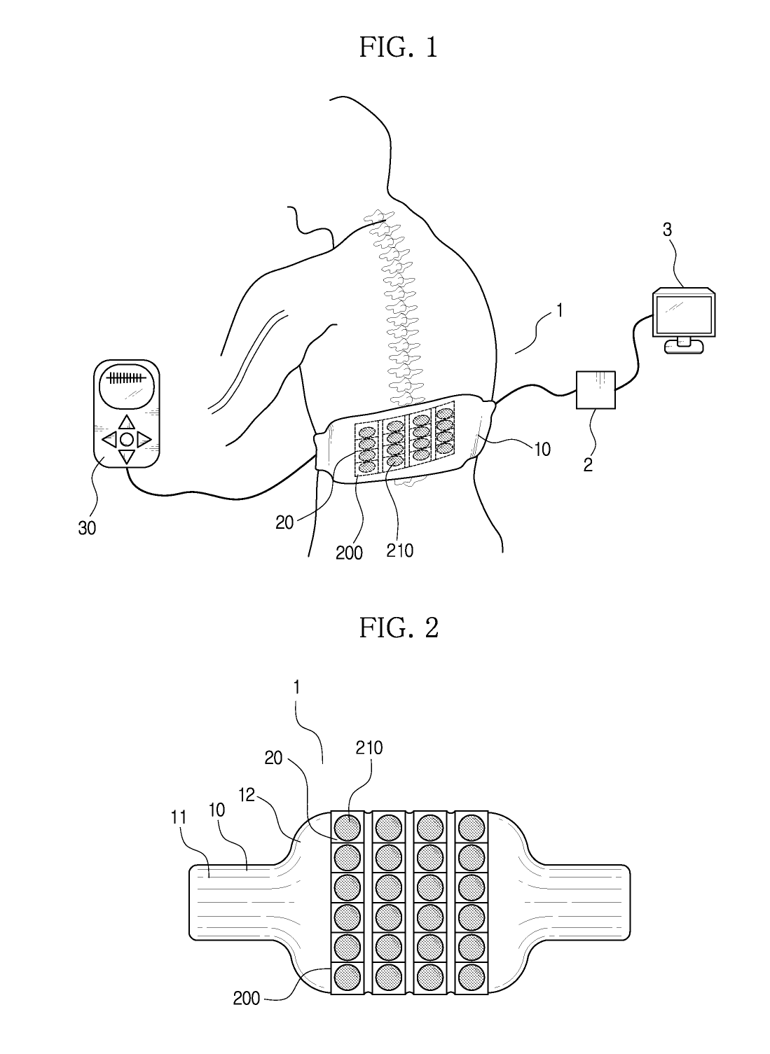

FIGS. 1 to 3 are diagrams for illustrating a stimulation apparatus 1 using low intensity focused ultrasound (hereinafter, referred to as an “ultrasound stimulation apparatus”) according to an embodiment of the present disclosure. FIG. 1 shows that the ultrasound stimulation apparatus 1 is attached to an upper body of a user.

The ultrasound stimulation apparatus 1 includes a low intensity ultrasound focusing array 20 to which a plurality of transducers 200 is attached, and a fixing device 10 to which the low intensity ultrasound focusing array 20 is attached. In FIG. 1, a reference symbol 30 designates a controller, and configuration and functions of the controller 30 will be described later.

A functional module 2 including a signal generator for generating a voltage signal applied to the transducer 200 and an amplifier for amplifying the signal may be connected to the ultrasound stimulation apparatus 1, and a computer 3 for controlling the functional module 2 and outputting various signals to a monitor may be further connected thereto.

The low intensity ultrasound focusing array 20 of this embodiment includes a plurality of transducers 200 arranged in a matrix shape. Each transducer 20 includes a piezoelectric element. In another case, the piezoelectric elements 210 may be arranged in a circular ring shape.

The piezoelectric element 210 of each transducer 200 outputs ultrasound having sound intensity lower the spatial-peak temporal-average sound intensity (Ispta) which is 3 W/cm², which does not damage a human body. The low intensity ultrasounds are overlapped to form a low intensity ultrasound beam.

The fixing device 10 of this embodiment has a shape of an abdominal binder which surrounds and binds an upper body of a user, and includes a fixing portion 12 to which the low intensity ultrasound focusing array 20 is attached and adhering portions 11 extending from both ends of the fixing portion 12 and capable of being coupled to each other.

In this embodiment, if the user wears the fixing device 10 having an abdominal binder shape on the upper body, the piezoelectric element 210 of the low intensity ultrasound focusing array 20 is closely fixed to the back of the user.

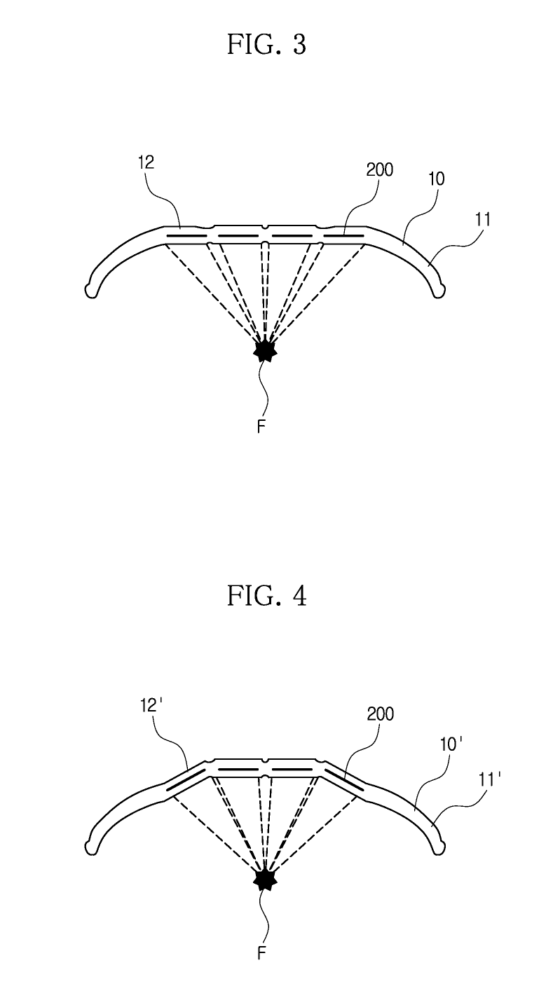

In this embodiment, the low intensity ultrasound beams output from the transducers 200 are focused to at least one focus F. FIG. 3 shows that the low intensity ultrasound beams output from the transducers 200 are focused to a single focus F.

All transducers 200 are disposed at a front portion, and in order to focus the low intensity ultrasound beams to a single focus F, a phase difference is given to ultrasounds having a square wave form generated by the piezoelectric elements 210. This will be described later in more detail.

Different from the above, it is also possible that low intensity ultrasound beams are focused to a single focus F by using the shape of the fixing device. FIG. 4 shows an ultrasound stimulation apparatus according to another embodiment of the present disclosure.

As shown in FIG. 4, different from the above embodiment, a fixing portion 12′ of a fixing device 10′ has a bent shape, instead of a flat shape, and the transducers 200 of the low intensity ultrasound focusing array are disposed at the fixing portion 12′ to emit low intensity ultrasound beams toward a single point. The adhering portion 11′ of this embodiment has the same configuration and operations as the adhering portion 11 of the former embodiment.

In this embodiment, even though a phase difference is not given to ultrasounds having a square wave form generated by the piezoelectric elements 210, low intensity ultrasound beams are focused to a single focus F. In addition, since the fixing portion 12′ is bent suitable for the upper body of the user, the user may wear the ultrasound stimulation apparatus more conveniently.

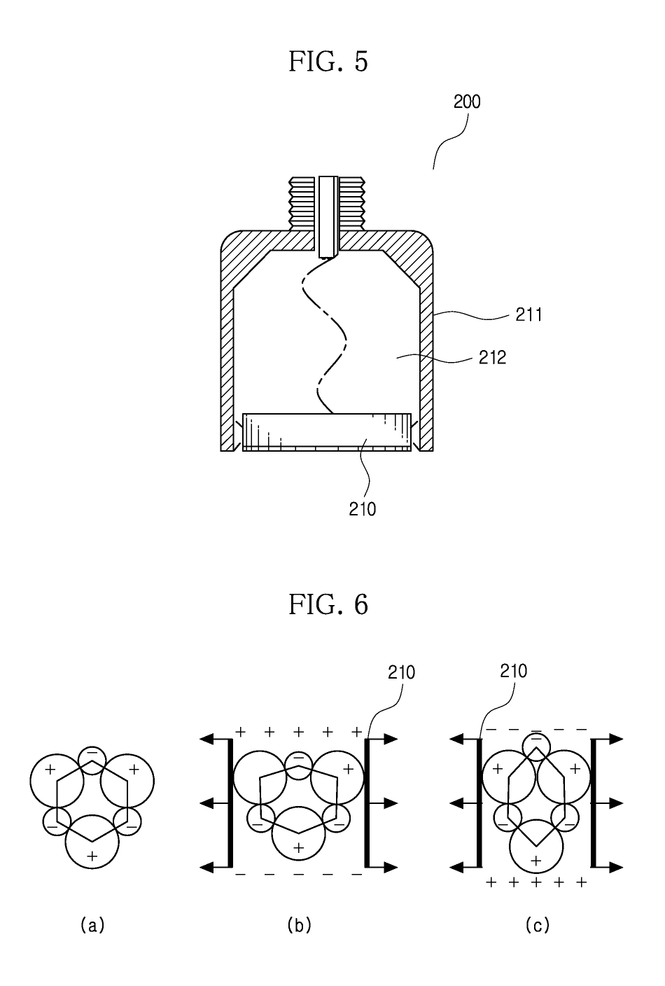

FIG. 5 is a diagram for illustrating a transducer 200 according to an embodiment of the present disclosure.

As shown in FIG. 5, the transducer 200 of this embodiment includes a body 211 having one open side and a piezoelectric element 210 formed in the opening of the body 211. The inside of the body 211 is filled with air 212. A cable is connected to each piezoelectric element 210 to apply a voltage to the piezoelectric element 210.

The body 211 may have a shape for fixing a single piezoelectric element 210 or may have an elongated shape to fix a plurality of piezoelectric elements 210 simultaneously.

In this embodiment, the piezoelectric element 210 is made of piezoelectric material such as quartz or turmaline, and the transducer 200 generates ultrasound by using the piezoelectric effect of the piezoelectric element 210.

FIG. 6 is a diagram for illustrating a piezoelectric effect of a piezoelectric element 210.

As shown in FIG. 6, if tension and compression are repeatedly applied along one axis of the piezoelectric element 210 made of quartz, a positive charge (+) is created at one side and a negative charge (−) is created at the other side, thereby generating an electric current.

The polarization effect at the piezoelectric element 210 is generated when a crystal structure is crushed and thus relative locations of (+) ion and (−) ion change. By doing so, the center of gravity of charge having a changed location in an element is automatically compensated, but an electric field is formed between both end surfaces of the crystal. The directions of the electric field at tension and compression are opposite to each other.

Inversely, if a voltage is applied to both end surfaces of the piezoelectric element 210, (+) ion in the electric field moves toward the (−) electrode, and (−) ion moves toward the (+) electrode. By means of such an inverse piezoelectric effect, the piezoelectric element 210 is guided to extend or shrink according to a direction of the voltage applied from the outside.

As the piezoelectric element 210 repeats extending and shrinking, ultrasound having a frequency equal to or greater than an audible range is generated in a similar way of a speaker.

As well shown in FIG. 2, the transducer 200 of this embodiment is a phased array transducer in which a plurality of piezoelectric elements 210 for individually receiving a voltage signal and outputting ultrasound is arranged.

As described above, the transducer 200 of this embodiment outputs a low intensity ultrasound beam by means of overlapping of ultrasounds output from the piezoelectric elements 210, and in this embodiment, the low intensity ultrasound beams output from the transducers 200 are focused to at least one focus F.

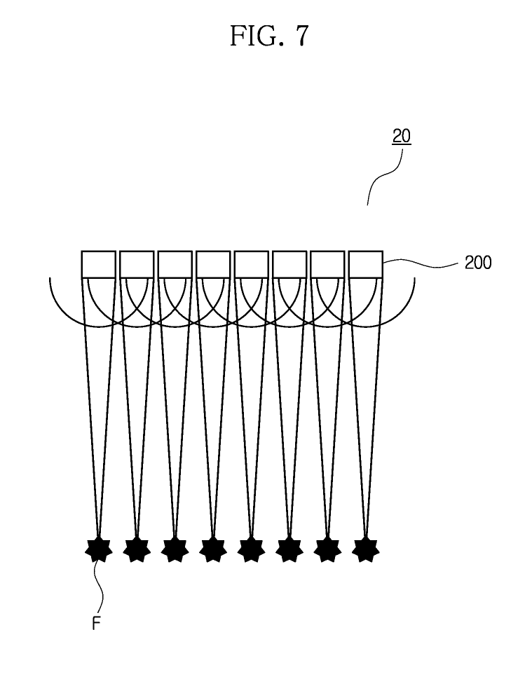

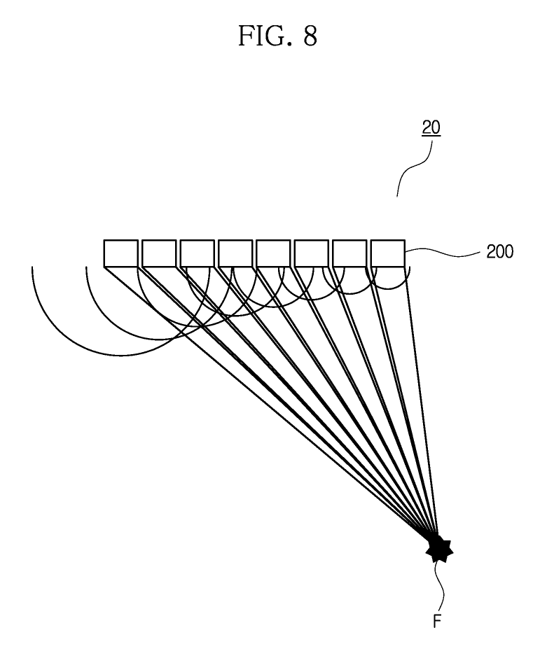

FIGS. 7 and 8 show a focused ultrasound beam.

As shown in FIG. 7, the piezoelectric elements 210 respectively generate square ultrasounds, and the square ultrasounds generated by the piezoelectric elements 210 are overlapped.

Due to the overlapping, a low intensity ultrasound beam may be formed at a focus F which is spaced apart from the transducer 200 by a predetermined distance.

FIG. 7 shows a case where a phase difference is not given to square waves generated by the piezoelectric elements 210, and here, ultrasound beams are respectively emitted from the piezoelectric elements 210 in a vertical direction toward the focus F.

Different from the above, as shown in FIG. 8, a phase difference may be given to the square ultrasounds generated by the piezoelectric elements 210, and in this case, the low intensity ultrasound beams may be focused to a single focus F.

In addition, if a phase difference among the square ultrasounds generated by the piezoelectric elements 210 is controlled, the location of the focus F may also be adjusted.

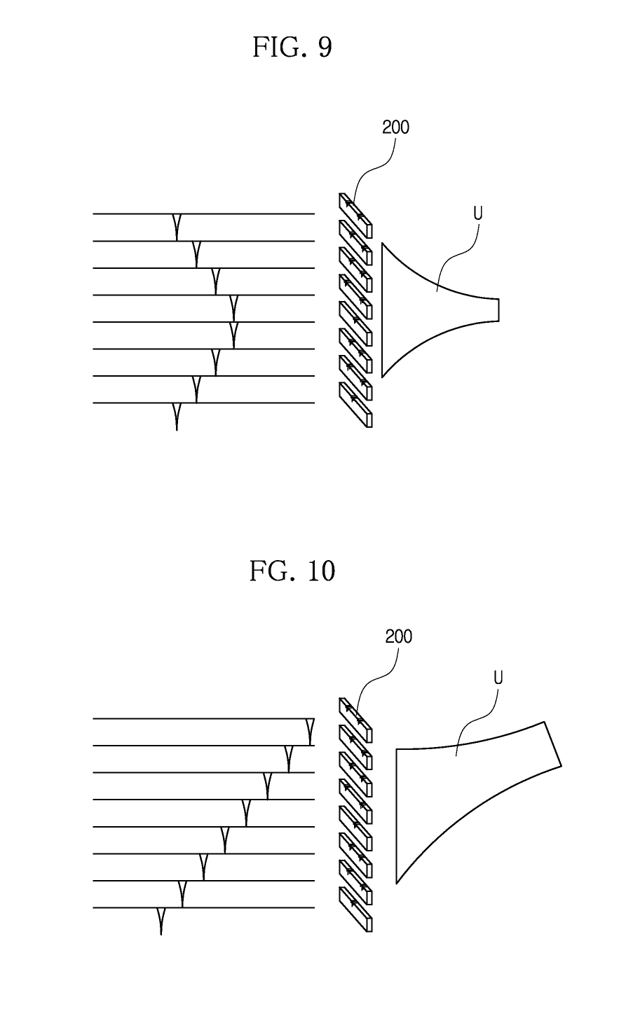

FIGS. 9 and 10 are showing that the location of the focus F is adjusted.

In FIGS. 9 and 10, a graph depicted in the left side shows a voltage signal applied to each transducer 200 with a time difference.

As comparatively shown in FIGS. 9 and 10, if a time difference is changed to the voltage signals respectively applied to the transducers 200, a phase difference among the square ultrasounds generated by the piezoelectric element 210 is varied, and also the location of the focus F is changed. The location of the focus F may be three-dimensionally adjusted in front and rear, right and left, and upper and lower directions.

There is a pain theory in which a single gate is present at a spinal cord and this increases or suppresses the transfer of a pain signal, which is a so-called “Gate control theory”. This theory supports many pain relieving methods such as spinal cord stimulation (SCS) for relieving a pain by means of acupuncture, behavior modification, electric stimulation or the like.

A pain signal is transferred by activating small nerve fibers and large nerve fibers in the spinal cord. The small nerve fiber represents a C-fiber which is smallest and has a low transfer speed among primary afferent fibers, and the large nerve fiber represents an A-delta fiber.

Projection cells are activated by the small nerve fiber and the large nerve fiber to transfer a pain signal through a spinothalamic tract of the brain. An inhibitory neuron is a kind of interneuron and is activated by the large nerve fiber. The neuron suppresses activations of projection cells to block the transfer of a pain signal.

If there is no stimulation from the outside, the inhibitory neuron blocks activation of projection cells. This means that the gate is closed.

If a general sensory signal is input, more large nerve fibers are activated than small nerve fibers. The large nerve fibers activate inhibitory neuron and projection cells. At this time, inhibitory neuron and projection cells are activated. At this time, the activated inhibitory neuron suppresses the transfer of a pain signal by the projection cells. In other words, this means that the gate is closed.

When a pain nerve signal is input, more small nerve fibers are activated than large nerve fibers. The activated small nerve fibers suppress inhibitory neuron and activate projection cells. Therefore, the projection cells transfer a pain signal to the brain without being affected by the inhibitory neuron. In other words, this means that the gate is open.

After the pain signal is transferred to the brain, the brain recognizing the pain activates inhibitory neuron as a feedback to suppress projection cells, thereby reducing the pain.

As described above, the pain is relieved based on the activation of nerve cells, and it has been proved by the study of Tyler researcher team that low intensity focused ultrasound allows activation of nerve cells.

In addition, a pain may be reduced by suppressing pain-related nerve signals of afferent nerve fibers of a patient, and a low intensity ultrasound technique for suppressing nerve signals of afferent nerve fibers may be used to reduce the pain.

Low intensity focused ultrasound gives a stimulation to a portion where a focus of the ultrasound beam is located.

In the ultrasound stimulation apparatus 1 of this embodiment, the focus F of the focused low intensity ultrasound beam is positioned at the spinal cord of the user, so that a point causing a pain to the user is found and also the corresponding point is intensively stimulated to relieve the pain.

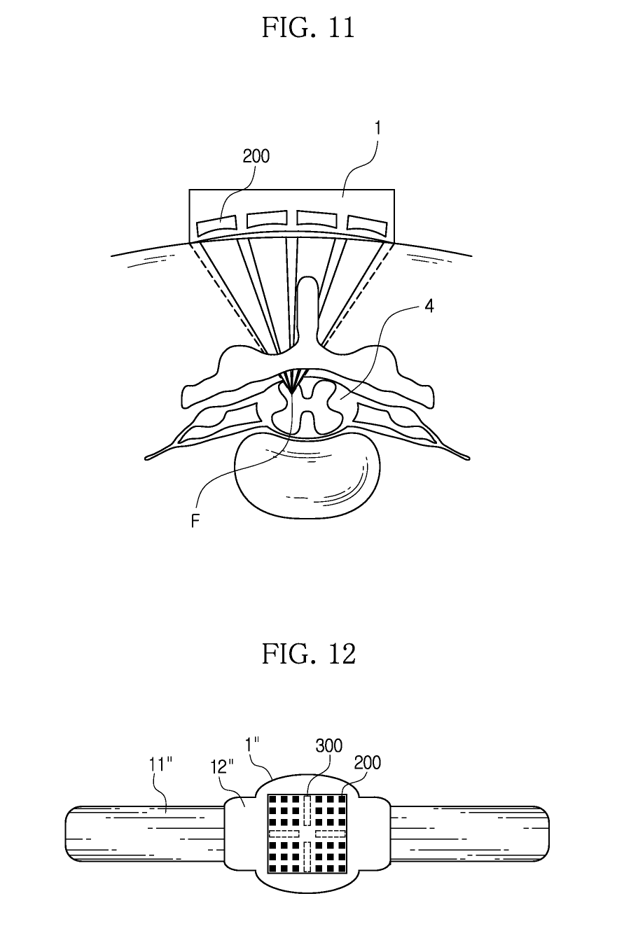

FIG. 11 is a diagram showing that a pain is controlled by stimulating a spinal cord 4 using the ultrasound stimulation apparatus 1.

The ultrasound stimulation apparatus 1 is adjusted so that an initial location of the focus F is on the spinal cord 4 when the transducer 200 is closely adhered to the back based on an average height of human.

Referring to FIG. 1, the ultrasound stimulation apparatus 1 includes a controller 30 which is operated by the user or another assistant such as a clinician to move the location of the focus F.

A clinician may expect a region of the spinal cord which serves as a gate of the pain of the user and set so that the location of the focus F is moved within the corresponding region.

Though not shown in detail, the controller 30 includes direction keys for adjusting the location of the focus F and number keys for setting the location of the focus F.

While continuously moving the location of the focus F within the spinal cord region set by the clinician by using the direction keys of the controller 30, the user may give low intensity ultrasound stimulation to the corresponding region. During this process, the user may feel that the pain is reduced.

If the user finds a point where the pain is reduced, the user may memorize or store the point where the pain is reduced, by putting the corresponding number keys of the controller 30.

Subsequently, while moving the location of the focus F successively, the user may repeat finding a point where the pain is reduced and storing the corresponding point with another number.

While moving within the spinal cord region set by a clinician, the user may give low intensity ultrasound stimulation to the corresponding region.

If a point where the pain is reduced is found by scanning the entire spinal cord region set by the clinician, the user pushes the stored number to automatically move the location of the focus F to a pain-reducing point corresponding to the number, thereby intensively treating and relieving the pain.

Different from the above, the clinician may store several points, which are expected as pain-reducing points, in advance, so that a point where the pain is reduced is found by intermittently moving the location of the focus F by pressing numbers one by one.

If there is a point where the pain is reduced, the user may press a number corresponding to the region to give ultrasound stimulation to the corresponding point.

Meanwhile, if a spinal cord and/or nerve cells connected to a muscle are stimulated, action potential is caused to propagate the stimulation to surrounding regions, which allows stimulation of muscular fibers and thus strengthening of the muscle.

The muscle strengthening is also generated based on activation of nerve cells, and nerve cells may be activated by means of low intensity ultrasound stimulation as described above.

FIG. 12 is a diagram showing that low intensity ultrasound stimulation is applied to nerve cells around a damaged spine using the ultrasound stimulation apparatus 1.

As shown in FIG. 12, a patient whose spine is fixed with a screw may not move easily and thus the muscle is weakened, which increases the possibility of secondary complications.

In this embodiment, low intensity ultrasound stimulation is performed to a spinal cord 4 near the damaged spine or nerves 5 around the spinal cord to support muscle strengthening around the damaged spine.

This is different from the pain control, since the nerves 5 around the spinal cord as well as the spinal cord 4 are stimulated.

In order to check the effect of the low intensity ultrasound stimulation for muscle strengthening and find optimal muscle strengthening stimulation point, the ultrasound stimulation apparatus 1″ according to another embodiment of the present disclosure further includes an electromyogram detecting device for measuring shrinkage or strengthen of muscle by means of electromygraphy (EMG).

The electromygraphy (EMG) is a method for diagnosing functional abnormality of muscle by measuring action potential caused by shrinkage of the muscle, and here, in the measurement of surface electromyogram, electrodes are attached to the skin surface to measure electromyogram, different from the needle electromyogram where electric activity of muscular movement is locally measured. The surface electromyogram is a painless non-invasive method, which allows quantitative measurement of a muscular movement group.

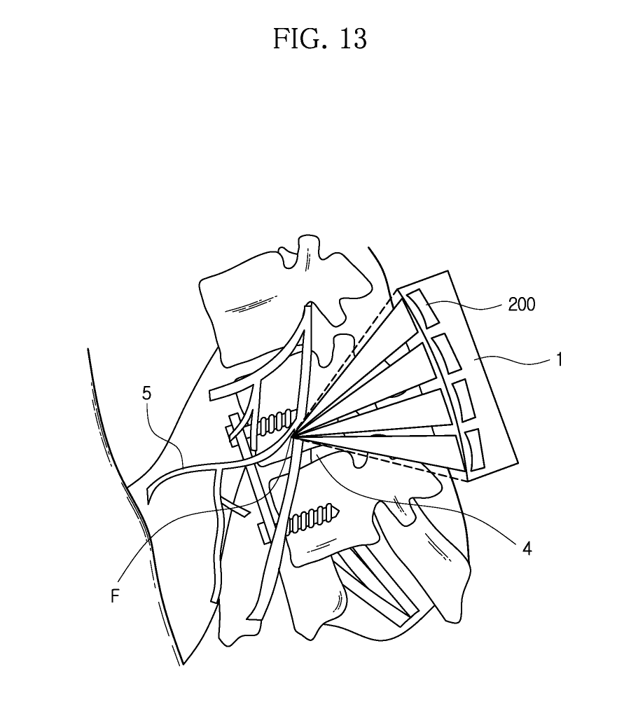

FIG. 13 is a diagram showing the ultrasound stimulation apparatus 1″ of this embodiment.

The ultrasound stimulation apparatus 1″ of this embodiment includes a surface electromyogram detecting device 300 for measuring electric activity of a muscle by performing the surface electromyogram with a fixing portion 12″ of the fixing device.

The transducer 200 of this embodiment has an independent form having a single piezoelectric element.

Four electromyogram detecting devices 300 are formed among the transducers 200 and disposed approximately at a center of the fixing portion 12″ in a cross shape to enhance the electromyogram efficiency.

The user locates the ultrasound stimulation apparatus 1″ on the back so that the electromyogram detecting device 300 comes into contact with the skin, and fixes the stimulation apparatus by using the adhering portion 11″.

The ultrasound stimulation region may be determined by a clinician through medical examination, and the ultrasound stimulation apparatus 1″ scans the corresponding region as a whole by moving the location of the focus F.

The user moves the location of the focus F continuously or intermittently by using the controller 30 and gives low intensity ultrasound stimulation to the spinal cord 4 near the damaged spine or the nerves 5 around the spinal cord.

At this time, the electromyogram detecting device 300 measures surface electromyogram of the user and displays the corresponding data through the monitor of the computer 3 (see FIG. 1) or a liquid crystal display of the controller 30.

The surface electromyogram measurement data may serve as an index for checking the degree of muscle shrinkage and muscle strengthening of the user. For example, if surface electromyogram increases over a threshold value at a specific stimulation location, the user or clinician may determine the corresponding location as a muscle strengthening stimulation point where muscle activity is vigorous and thus relevant muscles are reinforced.

If the low intensity ultrasound stimulation is completely given to the designated region to search all optimal muscle strengthening stimulation points, the user may press number keys of the controller 30 to store the point where the pain is reduced.

If the muscle strengthening stimulation point is completely searched, the location of the focus may be moved to the muscle strengthening stimulation point to intensively reinforce the muscle.

In this embodiment, the user or clinician finds a muscle strengthening stimulation point by checking the monitor, but the present disclosure is not limited thereto.

If the surface electromyogram increases over a threshold value at a specific stimulation location, the computer 3 may automatically determine and store the corresponding location as a muscle strengthening stimulation point, and if the searching work is completed, low intensity ultrasound stimulation may be applied to the searched muscle strengthening stimulation point without any operation of the user.

In this embodiment, the intensity of low intensity ultrasound beams may be differently set for various regions or stimulation points for muscle strengthening.

If the ultrasound stimulation apparatus 1 of this embodiment is used, low intensity ultrasound stimulation may be provided for pain relieving and muscle strengthening without damaging the human body.

In addition, due to excellent transportability and proximity, the ultrasound stimulation apparatus 1 of this embodiment may be conveniently used at home or hospitals.

What is claimed is:

1. A stimulation apparatus using low intensity focused ultrasound, comprising:
a low intensity ultrasound focusing array having a plurality of transducers for outputting low intensity ultrasound beams; and
a fixing device to which the low intensity ultrasound focusing array is attached, the fixing device being configured to fix the low intensity ultrasound focusing array to an upper body of a user,
wherein the low intensity ultrasound beams outputted from the transducers are focused to at least one focus, and
wherein the focus is positioned to a spinal cord of the user or nerves around the spinal cord so that low intensity ultrasound stimulation is applied to the spinal cord or nerve cells of the nerves around the spinal cord.

2. The stimulation apparatus using low intensity focused ultrasound according to claim 1,
wherein the low intensity ultrasound beams are focused to a single focus, and
wherein the position of the focus is three-dimensionally moved by adjusting a phase difference of low intensity ultrasound generated by the transducers.

3. The stimulation apparatus using low intensity focused ultrasound according to claim 2,
wherein low intensity ultrasound stimulation is applied to a preset region of the spinal cord by moving the location of the focus continuously or intermittently, so as to find a point where a pain of the user is reduced.

4. The stimulation apparatus using low intensity focused ultrasound according to claim 3,
wherein the point where the pain is reduced is stored, and
wherein if the low intensity ultrasound stimulation is completely applied to the preset region, the location of the focus is moved to the point where the pain is reduced, and then the pain of the user is intensively treated or relieved.

5. The stimulation apparatus using low intensity focused ultrasound according to claim 4, further comprising a controller which is operated by the user or another assistant to move the location of the focus.

6. The stimulation apparatus using low intensity focused ultrasound according to claim 2
wherein low intensity ultrasound stimulation is applied to a spinal cord near a damaged spine or nerves around the spinal cord by moving the location of the focus continuously or intermittently, so as to assist muscle strengthening near the damaged spine.

7. The stimulation apparatus using low intensity focused ultrasound according to claim 6,
wherein the fixing device includes a surface electromyogram detecting device for measuring electric activity of a muscle by means of surface electromyogram, and
wherein a muscle strengthening stimulation point where muscle strengthening is performed by the low intensity ultrasound is found by using the surface electromyogram measured by the electromyogram detecting device.

8. The stimulation apparatus using low intensity focused ultrasound according to claim 7,
wherein the found muscle strengthening stimulation point is stored, and
wherein, if the muscle strengthening stimulation point is found, the location of the focus is moved to the muscle strengthening stimulation point to intensively reinforce the muscle.

9. The stimulation apparatus using low intensity focused ultrasound according to claim 1,
wherein the fixing device is an abdominal binder surrounding an upper body of the user, and
wherein the abdominal binder includes:
a fixing portion to which the low intensity ultrasound focusing array is attached, and
adhering portions extending from both ends of the fixing portion and capable of being coupled to each other.

10. The stimulation apparatus using low intensity focused ultrasound according to claim 9,
wherein the fixing portion has a bent shape, and
wherein at the fixing portion, the transducers of the low intensity ultrasound focusing array are arranged to emit low intensity ultrasound beams toward one point.
